(19) United States
(12) Patent Application Publication
(10) Pub. No.: US 2011/0245192 A1
(43) Pub. Date: Oct. 6, 2011

(54) NEW ACTIVATORS FOR TREATING AND/OR PREVENTING DISEASES OR MEDICAL CONDITIONS WHICH BENEFIT FROM AN INCREASED TRANSPORT OF HYALURONAN ACROSS A LIPID BILAYER

(76) Inventor: Peter Prehm, Nottuln (DE)

(21) Appl. No.: 13/139,125

(22) PCT Filed: Dec. 14, 2009
(86) PCT No.: PCT/EP2009/067119

§ 371 Date: Jun. 10, 2011

(30) Foreign Application Priority Data
Dec. 12, 2008 (EP) 08171521.1
Sep. 11, 2009 (EP) 09170073.2
Sep. 25, 2009 (EP) 09171378.4

(51) Int. Cl. A61K 31/7034; C07C 233/54; C07H 15/203; A61K 31/196; A61P 17/06; A61P 17/10; A61P 17/02; A61P 17/00; A61P 11/00; A61K 8/02; A61K 8/60; A61K 8/42; A61Q 19/00

(52) U.S. Cl. 514/35; 562/453; 536/17.2; 562/455; 514/563

(57) ABSTRACT

The present invention relates in general to a compound (activator) which is characterized by a formula selected from the following formulas A, B and/or C or a pharmaceutically acceptable salt thereof. The present invention further relates to pharmaceutical composition comprising the activator(s) of the invention and to their use in the treatment of (for treating) and/or preventing diseases or medical conditions which benefit from an increased transport of hyaluronan across a lipid bilayer. The present invention also relates to a method for manufacturing a pharmaceutical composition comprising the steps of formulating the activator defined herein in a pharmaceutically acceptable form.

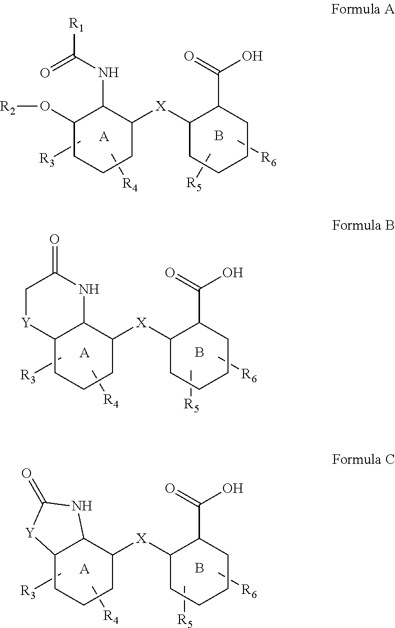

The present invention relates in general to a compound (activator) which is characterized by a formula selected from the following formulas A, B and/or C

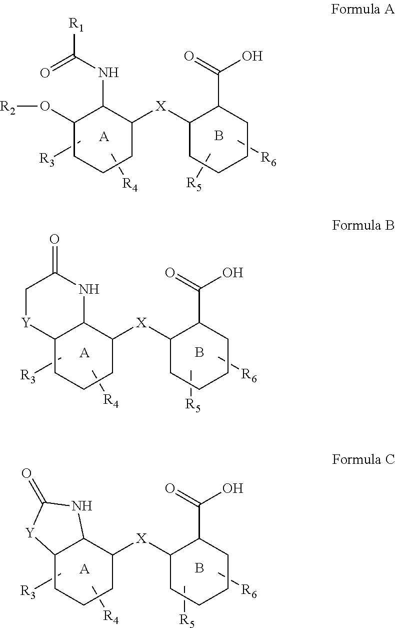

or a pharmaceutically acceptable salt thereof. The present invention further relates to pharmaceutical composition comprising the activator(s) of the invention and to their use in the treatment of (for treating) and/or preventing diseases or medical conditions which benefit from an increased transport of hyaluronan across a lipid bilayer. The present invention also relates to a method for manufacturing a pharmaceutical composition comprising the steps of formulating the activator defined herein in a pharmaceutically acceptable form.

A variety of documents is cited throughout this specification. The disclosure content of said documents (including any manufacturer's specifications, instructions etc.) is herewith incorporated by reference; however, there is no admission that any document cited is indeed prior art as to the present invention.

Hyaluronan is the major water binding component of the extracellular matrix. It is a very large glycosaminoglycan that is exported into the extracellular matrix by fibroblasts or epithelial cells, where it attracts water up to 99% of its own weight, swells to enormous volumes and displaces other resident macromolecules [1]. Typically, one molecule of hyaluronan with a molecular weight of 3.5 million Da totally occupies a sphere of about 440 nm [2]. Hyaluronan biosynthesis proceeds by alternate transfer of the precursor nucleotide sugars UDP-GlcA and UDP-GlcNac at the reducing end at the inner face of the plasma membrane [3-6]. The growing hyaluronan chain is synthesised within the cytoplasm and exported into the extracellular matrix where water attraction and swelling occurs. This mode of transmembrane transport was originally discovered in streptococci [7]. As the streptococcal hyaluronan transporter had structural and functional homology to human multidrug resistance transporters, we investigated hyaluronan exporters in human fibroblasts and identified MRP5 [8;9]. Our findings thus showed that two cellular processes are essential for the deposition of hyaluronan in the extracellular matrix, namely hyaluronan synthesis via the hyaluronan synthase in the cytosol and hyaluronan export through the plasma membrane via the MRP5 transporter.

In recent years it has become evident that cellular hyaluronan synthesis plays an important role in shedding and displacement of other components such as removal of antibodies or phagocytes from virulent Streptococci [10], detachment of fibroblasts during mitosis [11], tumour metastasis [12], as well as proteoglycan loss from osteoarthritic joints [13;14]. From all these observations, a new physiological function of hyaluronan can be postulated based on studies in several systems: Due to the enormous hydration volume, hyaluronan will replace any other components from its site of origin, when it extrudes from plasma membranes [1]. This concept may also apply for the rapid removal of mucus and adhering microorganisms from the bronchial epithelial surface that is pivotal for host defence.

The importance of hyaluronan for cellular behaviour had already been recognized for decades, but it was not until 1986 that the requirement for detachment in mitotic cell division was proven [11]. Hyaluronan was an adhesive cell surface component forming large coats around untransformed fibroblasts and smaller coats around transformed cells [15;16]. In humans, synthesis and degradation of hyaluronan is a very dynamic process. A total amount of hyaluronan of 34 mg is turned over in the circulation of an adult human daily [30;31]. The major origins of hyaluronan are joints, skin, eyes and intestine. The half-life in skin and joints is about 12 hours [32;33], in the anterior chamber of the eye it is 1-1.5 hours [34] and in the vitreous 70 days. The rapid turnover is surprising, because hyaluronan has been regarded as a structural component of the connective tissue.

The technical problem underlying the present invention is to provide means and methods for treating and/or preventing diseases or medical conditions which benefit from an increased transport of hyaluronan across a lipid bilayer.

The solution to this technical problem is achieved by providing the embodiments characterized in the claims.

It must be noted that as used herein, the singular forms “a”, “an”, and “the”, include plural references unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

The present invention relates, in general, to activators of the hyaluronan transport which are further specified herein below.

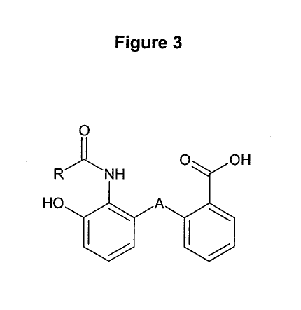

Starting from a lead structure (lead compound) which is depicted in FIG. 3, the present inventor were able to design several compounds (activators) which have the capability to enhance the hyaluronan transport/export in cells.

The lead compound for the design of the activators of the invention is also depicted below:

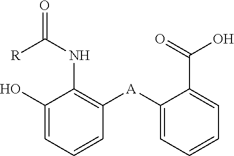

A further suitable starting point for the design of the activators of the invention is a compound which is exemplified by the formulas A, B or C as depicted below. These formulas might be seen as representatives of the “lead compound” of the invention.

This lead compound and/or its representatives can be converted into an activator of the invention by additional hydroxyl—or amino substituents—further possible exchanges are exemplified in great detail herein below. It is for example envisaged that the benzene rings can be connected by an oxygen, an amino (NH), a sulfur, a methylene (CH2), carbonyl (C═O) or an ester (—O—CO—) bridge. Optionally, the carboxyl group in ring B of the depicted formulas can be masked as an ester to prevent serious side effects due to stomach ulceration, a well known phenomenon for acidic nonsteroidal antirheumatic drugs (NSARD). These esters are readily cleaved by serum or cytosolic esterases to form the active acidic compound. The alcohol that forms the ester can carry additional functional groups such in nitric oxide releasing aspirin derivatives [260].

It is preferred that the activators of the present invention also obey the rule of 5 for “drugable” compounds:
There are not more than 5 H-bond donors (sum of OH and NH) in the molecule
There are no more than 10 H-bond acceptors (sum of N and O) in the molecule
The molecular weight does not exceed 500
Log P does not exceed 5
The PSA (Molecular polar surface area) does not exceed 150
   These features can conveniently be calculated by the skilled person, for example when using the information contained in the free website http://www.molinspiration.com/cgi-bin/properties. However, even without the information provided by hr referenced webpage, the skilled person is in a position to design an activator which obeys the above stated well-recognized rules of 5 for “drugable” compounds.

It thus follows that the present invention relates to compounds which are representatives of the lead compound of the invention and are, or were converted into, activators of the invention. “Activators of the invention” are described herein in great detail. The capabilities of these activators to enhance the hyaluronan transport/export are derivable from the appended examples and the tables disclosed herein.

The present invention, thus, relates to an activator which is characterized by a formula selected from the following formulas A, B and/or C

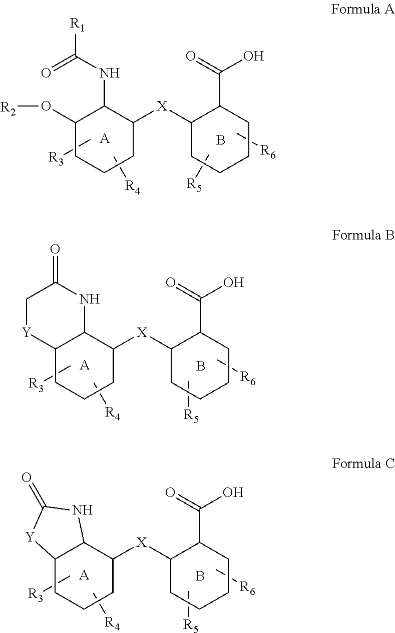

or a pharmaceutically acceptable salt thereof,

wherein

the ring systems A and B are independently selected from a monosaccharide, aryl (preferably phenyl), a heteroaryl or cycloalkyl (preferably cyclohexan), preferably with all substituents in equatorial configurations;

R1 is independently selected from alkyl (preferably C1 to C6), a substituted or unsubstituted phenyl, preferably CH3;

R2 is H, alkyl (preferably C1 to C6), a carbohydrate in a glycosidic β-linkage, preferably H;

R3, R4, R5, and R6 are independently selected from H, (OH) hydroxy, alkyl preferably C1 to C6, alkoxy (preferably C1 to C6), amino, alkylamino (preferably C1 to C6), halogen, benzylamino, or benzoylamino;
X is O, NH, alkylamino (NR), CO, S; and
Y is O, NH, alkylamino (NR), CO, S.

The term “benzylamino” refers to an amino group substitute with an benzyl group.

The term “benzoylamino” refers to an amino group substitute with an benzoyl group.

The terms “alkyl” and “alkylene” as used herein, whether used alone or as part of another group, refer to substituted or unsubstituted aliphatic hydrocarbon chains, the difference being that alkyl groups are monovalent (i.e., terminal) in nature whereas alkylene groups are divalent and typically serve as linkers. Both include, but are not limited to, straight and branched chains containing from 1 to about 12 carbon atoms, preferably 1 to about 6 carbon atoms, unless explicitly specified otherwise. For example, methyl, ethyl, propyl, isopropyl, butyl, i-butyl and t-butyl are encompassed by the term “alkyl.” Specifically included within the definition of “alkyl” are those aliphatic hydrocarbon chains that are optionally substituted. Representative optional substituents include, but are not limited to, hydroxy, oxo (=0), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN,—OH, oxo (=0), and amino groups.

The carbon number as used in the definitions herein refers to carbon backbone and carbon branching, but does not include carbon atoms of the substituents, such as alkoxy substitutions and the like.

The term “alkenyl”, as used herein, whether used alone or as part of another group, refers to a substituted or unsubstituted aliphatic hydrocarbon chain and includes, but is not limited to, straight and branched chains having 2 to about 10 carbon atoms (unless explicitly specified otherwise) and containing at least one double bond. Preferably, the alkenyl moiety has 1 or 2 double bonds. Such alkenyl moieties can exist in the E or Z conformations and the compounds of this invention include both conformations. Specifically included within the definition of “alkenyl” are those aliphatic hydrocarbon chains that are optionally substituted. Representative optional substituents include, but are not limited to, hydroxy, acyloxy, alkoxy,' amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Heteroatoms, such as O or S attached to an alkenyl should not be attached to a carbon atom that is bonded to a double bond. Preferred substituents include halogens, —CN, —OH, and amino groups

The term “alkynyl”, as used herein, whether used alone or as part of another group, refers to a substituted or unsubstituted aliphatic hydrocarbon chain and includes, but is not limited to, straight and branched chains having 2 to about 10 carbon atoms (unless explicitly 0 specified otherwise) and containing at least one triple bond. Preferably, the alkynyl moiety has about 2 to about 7 carbon atoms. In certain embodiments, the alkynyl can contain more than one triple bond and, in such cases, the alkynyl group must contain at least three carbon atoms. Specifically included within the definition of “alkynyl” are those aliphatic hydrocarbon chains that are optionally substituted. Representative optional substituents include, but are not limited to, hydroxy, \acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN, —OH, and amino groups Heteroatoms, such as O or S attached to an alkynyl should not be attached to the carbon that is bonded to a triple bond.

The term “cycloalkyl” as used herein, whether alone or as part of another group, refers to a substituted or unsubstituted alicyclic hydrocarbon group having 4 to about 7 carbon atoms, with 5 or 6 carbon atoms being preferred.

“Cyclohexane” is even more preferred.

Specifically included within the definition of “cycloalkyl” are those alicyclic hydrocarbon groups that are optionally substituted. Representative optional substituents include, but are not limited to, hydroxy, oxo (=0), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl.

The term “aryl”, as used herein, whether used alone or as part of another group, is defined as a substituted or unsubstituted aromatic hydrocarbon ring group having 5 to about 10 carbon atoms (unless explicitly specified otherwise) with 5 to 7 carbon atoms being preferred. The “aryl” group can have a single ring or multiple condensed rings. The term “aryl” includes, but is not limited to phenyl, a-naphthyl, (3-naphthyl, biphenyl, anthryl, tetrahydronaphthyl, fluorenyl, indanyl, biphenylenyl, and acenaphthenyl.

“Phenyl” is even more preferred.

Specifically included within the definition of “aryl” are those aromatic groups that are optionally substituted. In representative embodiments of the present invention, the, “aryl”groups are optionally substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. For example, the“aryl” groups can be optionally substituted with from 1 to 3 groups selected from Cl-C6 alkyl, Cl-C6 alkoxy, hydroxy, C3-C6 cycloalkyl, —(CH2)-C3-C6 cycloalkyl, halogen, Cl-C3 perfluoroalkyl, Cl-C3 perfluoroalkoxy, —(CH2) q-phenyl, and —O(CH2) q-phenyl. In these embodiments, the phenyl group of —(CH2) q-phenyl and —O(CH2) q-phenyl can be optionally substituted with from 1 to 3 groups selected from Cl-C6 alkyl, Cl-C6 alkoxy, phenyl, halogen, trifluoromethyl or trifluoromethoxy. In other embodiments, phenyl groups of the present invention are optionally substituted with from 1 to 3 groups selected from Cl-C6 alkyl, Cl-C6 alkoxy, —(CH2) p-phenyl, halogen, trifluoromethyl or trifluoromethoxy. Preferred aryl groups include phenyl and naphthyl. Preferred substituents on the aryl groups herein include alkyl, alkoxy, halo, cyano, nitro, trihalomethyl, and thioalkoxy

As used herein, the term “heteroaryl”, whether used alone or as part of another group, is defined as a substituted or unsubstituted aromatic heterocyclic ring system (monocyclic or bicyclic). Heteroaryl groups can have, for example, from about 3 to about 50 carbon atoms (unless explicitly specified otherwise), with from about 4 about 10 being preferred. In some embodiments, heteroaryl groups are aromatic heterocyclic ring systems having about 4 to about 14 ring atoms and containing carbon atoms and 1,2, or 3 oxygen, nitrogen or sulfur heteroatoms. Representative heteroaryl groups are furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3, 4-oxadiazole, 1,2, 4-triazole, 1-methyl-1, 2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5-or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from 0, N or S. Specifically included within the definition of heteroaryrare those aromatic heterocyclic rings that are substituted with 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. In some embodiments of the present invention, the “heteroaryl” groups can be optionally substituted with from 1 to 3 groups selected from Cl-C6 alkyl, Cl-C6 alkoxy, hydroxy, C3-C6 cycloalkyl, —(CH2)-C3-C6 cycloalkyl, halogen, Cl-C3 perfluoroalkyl, Cl-C3 perfluoroalkoxy, —(CH2) q-phenyl, and —O(CH2) q-phenyl. In these embodiments, the phenyl group of —(CH2) q-phenyl and —O(CH2) q-phenyl can be optionally substituted with from 1 to 3 groups selected from Cl-C6 alkyl, Cl-C6 alkoxy, phenyl, halogen, trifluoromethyl or trifluoromethoxy. Preferred heterocycles of the present invention include substituted and unsubstituted furanyl, thiophenyl, benzofuranyl, benzothiophenyl, indolyl, pyrazolyl, oxazolyl, and fluorenyl.

As used herein, the term“phenylcycloalkyl”, whether used alone or as part of another group, refers to the group Ra—Rb— wherein Rb is an optionally substituted cyclized alkyl group having from about 3 to about 10 carbon atoms with from about 3 to about 6 being preferred and Ra is an optionally substituted phenyl group as described above. Preferred cycloalkyl groups are cyclopropyl, cyclobutyl, cyclopentyl or cyclohexyl. Examples of phenylcycloalkyl also include groups of formula: EMI9.1 wherein R7 and R8 are, independently, hydrogen, Cl-C6 alkyl, Cl-C6 alkoxy, hydroxy, —(CH2) q-phenyl, —O(CH2) q-phenyl, C3-C6 cycloalkyl, halogen, Cl-C3 perfluoroalkyl or Cl-C3 perfluoroalkoxy; m is from 1 to 4, and q=0-6.

The term “alkoxy” as used herein, refers to the group Ra—O— wherein Ra is an alkyl group as defined above. Specifically included within the definition of “alkoxy” are those alkoxy groups that are optionally substituted. Preferred substituents on alkoxy and thioalkoxy groups include halogens, —CN,—OH, and amino groups

The term “arylalkyl” or “aralkyl” refers to the group —Ra—Rb, where Ra is an alkyl group as defined above, substituted by Rb, an aryl group, as defined above. Aralkyl groups of the present invention are optionally substituted. Examples of arylalkyl moieties include, but are not limited to, benzyl, 1-phenylethyl, 2-phenylethyl, 3-phenylpropyl, 2-phenylpropyl and the like.

The term “halogen” or “halo” refers to chlorine, bromine, fluorine, and iodine.

The term “alkylamino” refers to groups having the formula selected from: (a) —(CH2)m-NH2, where m=1 to 10, (b) —NH—(CH2)n-NH2, where n=1 to 10, or (c) —NH—(C2H4NH)xC2H4NH2, where x=0 to 5.

The term “monosaccharide” includes trioses like glyceraldehyde or dihydroxyacetone; tetroses like erythrose, threose or erythrulose; pentoses like arabinose, lyxose, ribose, deoxyribose, xylose, ribulose and xylulose; hexoses like allose, altrose, galactose, glucose, gulose, idose, mannose, fructose, psicose, sorbose tagatose and talose; heptoses like mannoheptulose, sedoheptulose; octoses like octolose, 2-keto-3-deoxy-manno-octonate or nonoses like sialose.

The term “carbohydrate” includes monosaccharides as defined above, disaccharides, or oligosaccharides consisting of 1 to 10, preferably 1 to 3 monosaccharides.

In a preferred embodiment, A and B are phenyl, R1 is methyl, R2 and R3 are hydrogen, R4 is hydroxyl, R5 is hydrogen and R6 is amino (compound 88)

In a further preferred embodiment, A is glucosamine and B are phenyl, R1 is methyl, R2 and R3 are hydrogen, R4, R5 and R6 are methoxy (compound 100)

In a preferred embodiment, the present invention relates to the compounds depicted below. The formulas of said compounds are depicted in the table below.

| | |
|---|---|
| Lead compound of the invention | 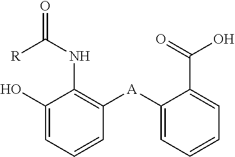 |
| Representative of the lead compound of the invention (Formula A) | 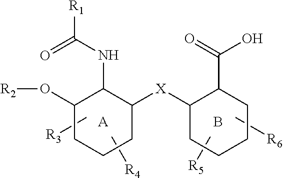 |
| Representative of the lead compound of the invention (Formula B) | 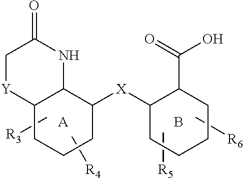 |
| Representative of the lead compound of the invention (Formula C) | 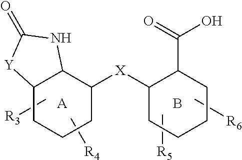 |
| Compound 89 | 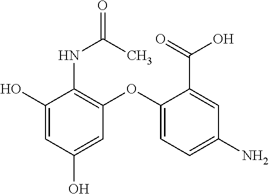 |
| Compound 101 | 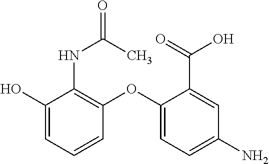 |
| Compound 100 | 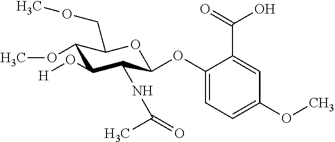 |
| Compound 86: | 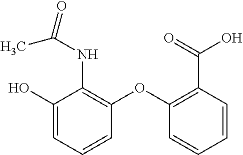 |
| Compound 87 | 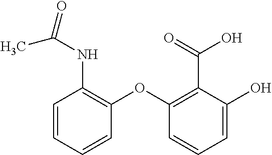 |
| Compound 90 | 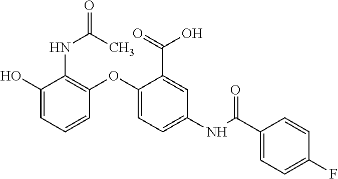 |

Compound 89 activates 4,7fold, while compound 101 activates 2,4fold. The “activatory capability” as described herein is expressed as the ratio of hyaluronan exported in the presence and absence of the compound. Methods to determine the hyaluronan transport are disclosed herein, and are also disclosed in great detail in WO2005/013947.

The present invention also relates to an inhibitor based on the above mentioned compounds. “Based on” means chemically altered derivatives, which derivatives have, preferably, a comparable biological function when compared with one of the compounds selected from the above depicted compounds, preferably with compound 89 or 101. “Comparable biological function” means that the chemical derivatives of the invention are able to increase the hyaluronan export with a deviation of the increasing activity in respect to one of the compounds selected from the above depicted compounds (preferably 89 or 101) of not more than about 40%, 30%, 20%, 15%, 10%, 5%, 2,5%, 2% or 1%, for example under conditions which equate to or are identical with those set out in Example 1. “Comparable biological function” does alternatively mean that the IC50 of the chemically altered derivatives of the invention deviates not more than about 40%, 30%, 20%, 15%, 10%, 5%, 2.5%, 2% or 1% from the IC50 of one of the compounds depicted above (preferably 89 or 101). WO2005/013947 discloses further suitable assays to evaluate the hyaluronan export.

Also included are the pharmaceutically acceptable salts of the activator(s) of the invention, including both organic and inorganic salts (e.g. with alkali and alkaline earth metals, ammonium, ethanolamine, diethanolamine and meglumine, chloride, hydrogen carbonate, phosphate, sulphate and acetate counterions). Appropriate pharmaceutically acceptable salts are well described in the pharmaceutical literature. In addition, some of these salts may form solvates with water or organic solvents such as ethanol. Such solvates are also included within the scope of this invention.

Furthermore, it has to be understood that the activator(s) of the present invention, can be further modified to achieve (i) modified organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state).

From the inhibitory profile of certain drugs (see appended Example 9 of WO2005/013947) and inhibitory-experiments with MRP5-specific RNAi (see Example 10 of WO2005/013947) it is evident that MRP5 is the most likely hyaluronan transporter.

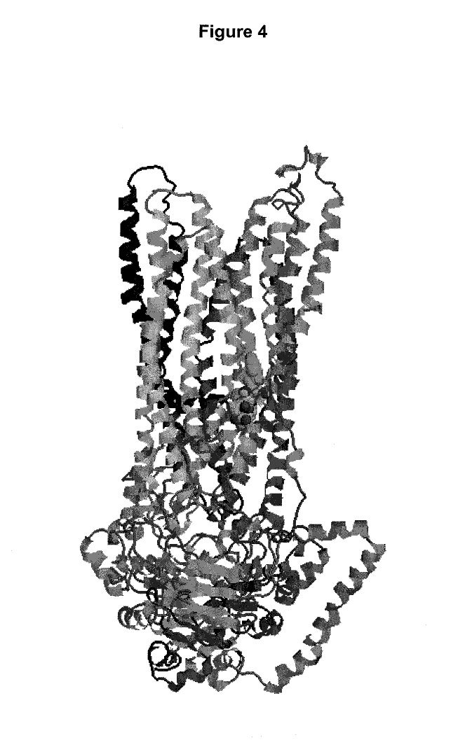

It is preferred that the activators of the present invention bind, preferably specifically, to the MRP5-transporter (see FIG. 4). MRP5 is an ABC-transporter which is described in great detail for example in WO2005/013947 (see for example the Examples 8 to 11 of WO2005/013947). A comprehensive recent review on ABC transporters is [143a]. The web-site http://www.nutrigene.4t.com/humanabc.htm also contains valuable information.

In a further preferred embodiment, it is envisaged that the activator of the invention increaes the hyaluronan transport rate to 5%, 10%, 20%, 30%, 40%, 50%, 60%, 70%, 80%, 90% or even 100% when compared to the transport rate that is achieved without the addition of said activator. One specific screening assay for the hyaluronan transporter is based on the extrusion of labelled hyaluronan oligosaccharides from intact cells in monolayer culture. Said assay is further explained in WO2005/013947, particularly in the appended examples of said document (e.g Example 8 or Example 11). In such cases it is sufficient to analyse the effect of the activator e.g. on a cell comprising MRP5, i.e. one compares the hyaluronan-transport before and after the addition of the activator and thereby identifies activators which increase the transport-rate of hyaluronan across a lipid bilayer.

In a preferred embodiment of the use or the methods of the present invention said activator(s) specifically increase(s) the transport of hyaluronan across a lipid bilayer mediated by MRP5. The term “specifically increase(s)” used in accordance with the present invention means that the activator specifically causes an increase of the transport of hyaluronan as mediated by MRP5 but has no or essentially has no significant effect on other cellular proteins or enzymes.

The activators can be discriminated by virtue of their binding to MRP5. Methods have been described that assay the binding of compounds to ABC transporters [92a;93a]. One specific screening assay for the hyaluronan transport as mediated by the ABC-transporter MRP5 is based on the extrusion of labeled hyaluronan oligosaccharides from intact cells in monolayer culture (see e.g. Example 11 of WO2005/013947). Alternatively, liposomes can be employed which encompass MRP5 in the lipid bilayer. For this assay, test-compounds like e.g. labeled hyaluronan oligosaccharides can be introduced into the cytosol of cells or into the liposomes. Because these test-compounds will normally not transverse the plasma membranes/lipid bilayer, they are introduced e.g. by osmotic lysis of pinocytotic vesicles according to a method that has already successfully been applied for the introduction of periodate oxidized nucleotide sugars [25a]. Alternatively, it is possible to introduce the test-compounds by other suitable methods like electro-chemical-poration; lipofection; bioballistics or microinjection (these methods are well-known in the art). Hyaluronan oligosaccharides are prepared from commercially available hyaluronan by digestion with hyaluronidase and sized fractionation by gel filtration as described [102a]. Appropriate oligosaccharide fractions having a length between 2 and 50 disaccharide units are labeled by incorporation of a biotin, radioactivity, or a fluorescent probe. These methods are routine published procedures [87a,99a-101a,103a]. For example the cells are seeded into multiwell microtiter plates to a density of at least 4×10⁴ cells/cm². When the cells are attached to the plastic surface after a few hours, they are washed with phosphate buffered saline and incubated with the labeled hyaluronan dissolved in medium for osmotic lysis of pinocytotic vesicles (growth medium such as Dulbeccos medium containing 1 M sucrose, 50% poly(ethylene glycol)-1000) for at least 5 min up to several hours at 37° C. During this time the cells will pinocytose this hyperosmotic medium and the labeled hyaluronan. The above medium is substituted by a mixture of Dulbeccos medium and water (3:2) for 2 min. This causes the intracellular pinocytotic vesicles to lyse and to liberate the contents into the cytosol without damaging the cells. The cells can be subjected to this incubation sequence several times. The cells are washed thoroughly several times with phosphate buffered saline or growth medium to remove extracellular labeled hyaluronan and are then ready for the assay. They are incubated in growth medium containing the compound to be tested in different concentrations for several hours. During this time the labeled hyaluronan will be transported back into the medium. The amount of labeled hyaluronan oligosaccharide in the medium can be determined by a biotin-related assay, by radioactivity or by fluorescence intensity.

For medical treatment it is advantageous to use activators that act in a reversible manner.

The activators of the invention may be employed for the preparation of a pharmaceutical composition for treating and/or preventing diseases or medical conditions which benefit from an increased transport of hyaluronan across a lipid bilayer. Such diseases/medical conditions are explained herein.

The pharmaceutical composition of the present invention may optionally comprise a pharmaceutical carrier.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use.

Upon using the activators of the present invention, it is possible to treat/ameliorate and/or prevent diseases or medical conditions which are characterized by a reduced hyaluronan export/transport and/or which benefit from an increased transport of hyaluronan across a lipid bilayer.

The skilled person is well aware which specific diseases are characterized by a reduced level of hyaluronan at the exterior of cells and, provided with the teaching and disclosure of the present invention can easily test for such a reduced hyaluronan transport. Thus, it is possible to identify a subject at risk for a disease which is associated with a reduced transport of hyaluronan across a lipid bilayer or to diagnose a disease which is associated with a reduced transport of hyaluronan across a lipid bilayer. This can be diagnosed e.g., by isolating cells from an individual. Such cells can be collected from body fluids, skin, hair, biopsies and other sources as described herein elsewhere. It is likewise known to the skilled person, which medical conditions will benefit from an increased transport of hyaluronan.

The term “reduced transport of hyaluronan” as used herein means that the transport of hyaluronan is below the transport level as compared with a normal/natural state of a comparable control-cell/subject. It has to be understood that in the context of the present invention, the terms “transport” and export” are used interchangeably.

“A disease which is characterized by/associated with a reduced transport of hyaluronan” means in general that the disease is characterized by (is attended by) an abnormal low production of hyaluronan and/or by the abnormal absence of hyaluronan in cells, tissues and/or body fluids. This can be determined e.g., by isolating cells from an individual and or by evaluating the presence of hyaluronan otherwise (e.g. by help of antibodies directed against said molecule or by way of ELISA-assays which are able to evaluate the content of hyaluronan etc.). Such cells can be collected from body fluids, skin, hair, biopsies and other sources as described herein elsewhere.

Deficient hyaluronan export by MRP5 should lead to intrauterine death, because the lack of hyaluronan deposition in the extracellular matrix is incompatible with life as demonstrated by hyaluronan synthase deficient mice, which die at a stage E9.5 during embryonic development (Camenisch et al., 2000, J Clin Invest, 106, 349-360).

Lower hyaluronan synthesis has been associated in the skin of patients with psoriasis and acne. These patients can be treated with retinoic acid containing drugs. Retinoic acid is known to induce the expression of the hyaluronan synthase and thus contributes to higher hyaluronan levels. These levels could also be stimulated with activators of hyaluronan export.

A disease or medical condition which “benefits from an increased transport of hyaluronan” refers to diseases which might benefit from an increased transport of hyaluronan (for example cystic fibrosis, psoriasis, acne, aged) and/or to medical conditions which are normally/naturally/frequently associated/characterized with an increased hyaluronan transport/export and/or an increased amount/concentration of hyaluronan per se (for example wound healing or scar-less healing).

The activators of the present invention are therefore useful for the medical treatment of acne, psoriasis, intrauterine death, wound healing, cystic fibrosis, and/or scar-less healing.

It is expected that these diseases/medical conditions will benefit from the activators of the present invention, as these activators could be used to activate the hyaluronan transport at an early (earlier) stage (for example before the “naturally occurring” increase of hyaluronan in response to a medical condition as exemplified herein) and/or to enhance the “naturally occurring” hyaluronan transport in response to a medical condition.

The term “increased/enhanced transport of hyaluronan” as used herein means that the transport of hyaluronan increases (leading to an increased amount of hyaluronan in the exterior/proximity of the respective cell(s)), depending on whether the activator was applied.

The term “activator” defines in the context of the present invention a compound as described herein. It is envisaged that the activators of the present invention are capable to enhance or initiate the transport of hyaluronan across a lipid bilayer in a cell/subject.

The term “normal/natural state” of a comparable control-cell/subject means the transport-rate of hyaluronan in a control-cell which is preferably of the same nature as the test-cell (e.g. both cell are chondrocytes) but which is derived from a different source.

“A different source” includes e.g. a cell/tissue sample obtained from a healthy subject which does not suffer from a disease which is associated with a reduced transport of hyaluronan across a lipid bilayer or a cell/tissue sample obtained from a distinct part/location of the same subject wherein said different part/location appears to be free from associated symptoms of a disease which is associated with a reduced transport of hyaluronan across a lipid bilayer. Assays and histological methods to classify a disease which is associated with a reduced transport of hyaluronan across a lipid bilayer are well-known to the skilled person (see for example WO2005/013947). However, even in cases where the activator will not increase the hyaluronan-transport across a lipid-bilayer to the normal/natural state of a comparable control-cell/subject but actually increases the hyaluronan transport when compared to the transport rate before the addition of said activator, it will be appreciated that said activator has a beneficial effect.

The term “increased” as used herein defines the increase of the hyaluronan transport across a lipid bilayer, for example to at least about the same level as compared to a normal/natural state of a comparable control-cell/subject.

Accordingly, it is envisaged that the activator of the invention at least increases the hyaluronan transport rate about 5%, 10%, 20%, 30%, 40%, 50%, 60%, 70%, 80%, 90% or even 100% when compared to the transport rate that is achieved without the addition of said activator. A suitable test system to measure the export/transport of hyaluronan is disclosed in the appended examples. Further test systems are disclosed in WO2005/013947.

The present invention relates in one embodiment to the activators as defined herein before as active compounds in a pharmaceutical composition.

It is also envisaged that the activators of the present invention are used for treating and/or preventing diseases or medical conditions which benefit from an increased transport of hyaluronan across a lipid bilayer.

It is also envisaged that the activators of the present invention (as defined herein before) are used for the preparation of a pharmaceutical composition for the treatment/ for treating and/or preventing diseases or medical conditions which benefit from an increased transport of hyaluronan across a lipid bilayer.

The term “lipid bilayer” is well-known to the skilled person [91a] and denotes e.g. biological membranes or liposomes. Assay and test-systems which allow the determination of hyaluronan-transport across a lipid bilayer are explained in the appended examples. It will be understood that the term “capable of transporting hyaluronan across a lipid bilayer” defines in the context of cells or tissues comprising said cells, the transport of hyaluronan to the exterior of the cell (e.g. the extracellular milieu of the respective cell).

The main hyaluronan producing cells in the body are fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, and epithelial cells. Chondrocytes represent only 5% of the tissue but they are responsible for synthesizing and controlling the matrix (including the hyaluronan production).

It is therefore envisaged that the activators of the present invention are used for the treatment of (for treating) diseases or medical conditions which benefit from an increased transport of hyaluronan across a lipid bilayer of the cells mentioned above.

The increase which is achieved by the activators of the present invention will also depend on the dosage and on the way of administration of the activator. The dosage regimen utilizing the activator of the present invention is therefore selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It has to be understood that in the context of the present invention, “an activator” includes “at least one activator”, wherein the term “at least one activator” comprises at least one, at least two, at least three, at least four, at least five, at least six . . . etc. activator(s) of the invention. It will be understood that the number of activators which are used together (simultaneously or displaced) will be selected on a case to case basis in order to provide a suitable treatment for the cell/tissue/subject. In this context, “suitable” means that the treatment with the respective activator(s) of the invention exerts a beneficial effect, e.g. it prevents, counters or arrests the progress of the condition.

The activators of the present invention can be applied prophylactically. Prophylactic treatment will be especially important for wound covering unguents or creams.

Thus in a further embodiment of the medical uses of the present invention said activator(s) is(are) to be administered prophylactically.

Alternatively, the activators can by applied therapeutically, preferably as early as possible.

Thus, in another embodiment of the medical uses of the present invention said activator(s) is(are) to be administered therapeutically.

The dosage regimen utilising the activators of the present invention is selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It is preferred that the activators of the invention are used in a therapeutically effective amount/concentration, i.e. in an amount/concentration that is sufficient to exert its activatory effect. Said amount/concentration can be determined by the methods disclosed in the appended examples. It is envisaged that the therapeutically effective amount/concentration of activator of the invention at least increases the hyaluronan transport rate about 5%, 10%, 20%, 30%, 40%, 50%, 60%, 70%, 80%, 90% or even 100% when compared to the transport rate that is achieved without the addition of said activator.

It is also envisaged that the activators of the present invention are employed in co-therapy approaches, i.e. in co-administration with other medicaments or drugs.

The present invention also relates to a method of preventing, ameliorating and/or treating the symptoms of a disease or medical conditions which benefit(s) from an increased transport of hyaluronan across a lipid bilayer in a subject comprising administering at least one activator as defined herein to the subject.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

In the context of the present invention the term “subject” means an individual in need of a treatment of an affective disorder. Preferably, the subject is a mammalian, particularly preferred a human, a horse, a camel, a dog, a cat, a pig, a cow, a goat or a fowl.

The term “administered” means administration of a therapeutically effective dose of the activators disclosed herein. By “therapeutically effective amount” is meant a dose that produces the effects for which it is administered. The exact dose will depend on the purpose of the treatment, and will be ascertainable by one skilled in the art using known techniques.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments. The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound(activator) or drug(activator) as defined herein, a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

Retinoic acid is known to enhance the hyaluronan synthase activity and, thereby, to improve the attractiveness of the skin (for example reduce wrinkles, skin smoothing etc.). Some cosmetics, therefore, have retinoic acid as an active ingredient. There are also natural products such as oel from the seed of rose hip containing high concentrations of retinoic acid. This oel is sold as skin smoothing cosmetic. It is, therefore, expected that the activators of the present invention are likewise suitable for enhancing the attractiveness of the skin as they promote the hyaluronan transport.

The invention is further directed to the cosmetic use of the compounds according to the invention for the preparation of a composition for enhancing the attractiveness of the skin (e.g. abate visible skin aging symptoms like wrinkles etc.).

The term “cosmetic use” comprises the use of the active compound according to the invention in cosmetic compositions; such as care products for the skin. The cosmetic compositions include for example skin cosmetic preparations, such as W/O or O/W skin and body creams, day and night creams, light protection compositions, aftersun products, skin aging products, hand care products, face creams, multiple emulsions, gelees, microemulsions, liposome preparations, niosome preparations, antiwrinkle creams, face oils, lipogels, sportgels, moisturizing creams, bleaching creams, vitamin creams, skin lotions, care lotions, ampoules, aftershave lotions, preshaves, humectant lotions, tanning lotions, cellulite creams, depigmentation compositions, massage preparations, body powders, face tonics, deodorants, antiperspirants, nose strips, antiacne compositions, repellents and others.

The term “skin aging” as used in the context of the invention, includes the so-called “intrinsic” and “extrinsic” aging of the skin. The biological mechanism of said aging of the skin is characterized by an alteration of the dermis with appearance of folds and wrinkles, sagging and relaxing of the cutaneous tissue.

The main signs of skin aging are the following:
(a) Appearance of deep wrinkles, increasing with age. A disorganization of the “grain” of the skin is noted, that is to say the micro-relief is less regular and is anisotropic in nature.
(b) The skin color is generally modified, appearing paler and yellower, which appears to be due chiefly to a disorganization of the microcirculation (less haemoglobin in the papillary layer of the dermis). Numerous colored spots appear at the surface, which is due to impaired melanogensis. On some areas, diffuse irritation and sometimes telangiectasia are present.
(c) Another sign of skin aging is the dry and rough appearance of the skin, which is due chiefly to greater desquamation, these squamae contributing also to the somewhat grey appearance of the color by diffracting light rays.
(d) Finally, a loss is noted in firmness and tonus of the skin, which, as in the case of wrinkles, is explained at least partially by a dermal and epidermal atrophy as well as a flattening of the dermoepidermal formation.

Thus, as used herein “skin aging” means at least one sign selected from the signs explained above, i.e. selected from (a) appearance of deep wrinkles, (b) modification of color of the skin, (c) dryness and roughness of the skin and/or (d) a loss is noted in firmness and tonus of the skin.

Accordingly, a further embodiment the invention is directed to a cosmetic composition comprising a compound of the invention as the active compound and a cosmetically acceptable carrier or excipient. It is also envisaged that the cosmetic composition of the invention contains further active substances which are known to enhance the attractiveness of the skin (for example retinoic acid).

The cosmetic composition may be delivered in various ways, such as topically. Topical administration of the cosmetic composition of the present invention is useful when the desired treatment involves areas or organs readily accessible by topical administration. For application topically to the skin, the cosmetic composition may be formulated with a suitable lotion, cream, gel, paste, ointment, or transdermal patches. The cosmetic can, depending on the field of use, also be in the form of a spray (pump spray or aerosol), foam, gel spray, mousse, suspensions or powders.

The cosmetic composition may be formulated with a suitable lotion or cream comprising the active components suspended or dissolved in a carrier. Such carriers include, but are not limited to, one or more of mineral oil such as paraffin, vegetable oils such as castor oil, castor seed oil and hydrogenated castor oil, sorbitan monostearate, polysorbate, fatty acid esters such as cetyl ester, wax, fatty acid alcohols such as cetyl alcohol, stearyl alcohol, 2-octyldodecanol, benzyl alcohol, alcohols, triglycerides and water.

Alternatively, the cosmetic composition may also be formulated with a suitable gel comprising the active components suspended or dissolved in a carrier. Such carriers include, but are not limited to, one or more of water, glycerol, propylene glycol, liquid paraffin, polyethylene, fatty oils, cellulose derivatives, bentonite and colloidal silicon dioxide.

Suitable propellants for aerosols according to the invention are the customary propellants, for example propane, butane, pentane and others.

A suitable paste comprises the active compound suspended in a carrier. Such carriers include, but are not limited to, petroleum, soft white paraffin, yellow petroleum jelly and glycerol.

The cosmetic composition may further comprise additional components, as are customarily used in such preparations, e.g. moisturizing substances, olfactory agents, emulsifiers, preservatives, perfumes, antifoams, dyes, pigments, thickeners, surface-active substances, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents, silicone derivatives, UV-filtering substances, or substances which absorb UV radiation in the UV-B and/or UV-A region.

The cosmetic composition according to the invention may preferably comprise moisturizing substances or emollients. Moisturizing substances or emollients may be used in amounts, which are effective to prevent or relieve dryness. Useful moisturizing substances or emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; ether esters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical moisturizing substances or emollients include mineral oil, especially mineral oils having a viscosity in the range of 50 to 500 SUS, lanolin oil, mink oil, coconut oil, cocoa butter, olive oil, almond oil, macadamia nut oil, aloa extract, jojoba oil, safflower oil, corn oil, liquid lanolin, cottonseed oil, peanut oil, purcellin oil, perhydrosqualene (squalene), caster oil, polybutene, odorless mineral spirits, sweet almond oil, avocado oil, calophyllum oil, ricin oil, vitamin E acetate, olive oil, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricinoleates of alcohols and polyalcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

Moreover, the cosmetic composition according to the invention may preferably comprise emulsifiers. Emulsifiers (i.e., emulsifying agents) are preferably used in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty acid monoesters containing sulfuric esters, e.g., glyceryl monostearate containing sodium lauryl surfate; (ii) cationics chloride such as N(stearoyl colamino formylmethyl) pyridium; N-soya-N-ethyl morpholinium ethosulfate; alkyl dimethyl benzyl ammonium chloride; diisobutylphenoxyethoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and ethoxylated cholesterol. The selection of emulsifiers is exemplarly described in Schrader, Grundlagen and Rezepturen der Kosmetika, Hüthig Buch Verlag, Heidelberg, 2^{nd} edition, 1989, 3^{rd} part.

The cosmetic composition of the present invention may preferably comprise a preservative. Preservatives used in compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

A cosmetic composition according to the invention may also comprise an olfactory agent or perfume. Olfactory agents, perfumes (fragrance components) and colorants (coloring agents) well known to those skilled in the art may be used in effective amounts to impart the desired fragrance and color to the compositions of the invention

The cosmetic composition according to the invention may also include a surfactant. Suitable surfactants may include, for example, those surfactants generally grouped as cleansing agents, emulsifying agents, foam boosters, hydrotropes, solubilizing agents, suspending agents and non-surfactants (facilitates the dispersion of solids in liquids).

The surfactants are usually classified as amphoteric, anionic, cationic and non-ionic surfactants. Amphoteric surfactants include acylamino acids and derivatives and N-alkylamino acids. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic acids; sulfonic acids and salts, such as, acyl isothionates, alkylaryl sulfonates, alkyl sulfonates, and sulfosuccinates; sulfuric acid esters, such as, alkyl ether sulfates and alkyl sulfates. Cationic surfactants include: alkylamines, alkyl imidazolines, ethoxylated amines, and quaternaries (such as, alkylbenzyldimethylammonium salts, alkyl betaines, heterocyclic ammonium salts, and tetra alkylammonium salts). Nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

Furthermore, a cosmetic composition according to the invention may also comprise a film former. Suitable film formers which are used in accordance with the invention keep the composition smooth and even and include, without limitation: acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like. Generally, film formers can be used in amounts of about 0.1% to about 10% by weight of the total composition with about 1% to about 8% being preferred and about 0.1 DEG/O to about 5% being most preferred.

Humectants can also be used in effective amounts, including: fructose; glucose; glutamic acid; glycerin; honey; maltitol; methyl gluceth-10; methyl gluceth-20; propylene glycol; sodium lactate; sucrose; and the like.

Compositions according to the invention may be prepared according to methods well known to the person of ordinary skills in the art (see e.g. Bauer et al., Pharmazeutische Technologie, 5. edt. Govi-Verlag Frankfurt, 1997; Rudolf Voigt, Pharmazeutische Technologie, 9. edt., Deutscher Apotheker Verlag Stuttgart, 2000).

A cosmetic composition according to the invention comprises, for example 0/W and W/O creams, O/W and W/O emulsions, gels, multiple emulsions (W/O/W and O/W/O), cosmetic dispersions (hydrodispersions and lipodispersions), sticks, formulations comprising a tenside or simple solutions (oily or aqueous).

An O/W formulation for the skin may be formulated by mixing, for example, the following ingredients in accordance with the International Nomenclature of Cosmetic Ingredients, INCI:
A ceteareth-6, stearyl alcohol, ceteareth-25, diethylamino hydroxybenzoyl hexyl benzoate, PEG-14 dimethicone, cetearyl alcohol, ethylhexyl methoxycinnamate, dibutyl adipate;
B glycerol, panthenol, preservative, aqua dem;
C caprylic/capric triglyceride, sodium acrylates copolymer;
D sodium ascorbyl phosphate, tocopheryl acetate, bisabolol, caprylic/capric triglyceride, sodium ascorbate, tocopherol, retinol;
   active compound; and
E sodium hydroxide

Phases A and B are separately heated. Phase B is subsequently stirred into phase A and homogenized. Phase C is stirred into a combination of phases A and B and homogenized. The mixture is under agitation cooled down; then phase D is added and the pH is adjusted with phase E. The solution is subsequently homogenized and cooled down to room temperature.

The exact amount of the particular ingredients and conditions may vary dependent on the particular application and administration form. The person skilled in the art is able to easily determine the exact amount and condition given the specification and references therein.

This disclosure may best be understood in conjunction with the accompanying drawings, incorporated herein by references. Furthermore, a better understanding of the present invention and of its many advantages will be had from the following examples, given by way of illustration and are not intended as limiting.

The figures show:

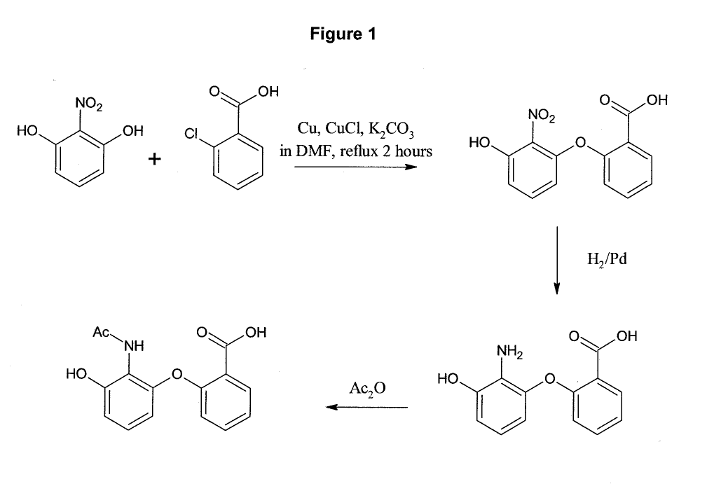

FIG. 1 Chemical synthesis of compound 86

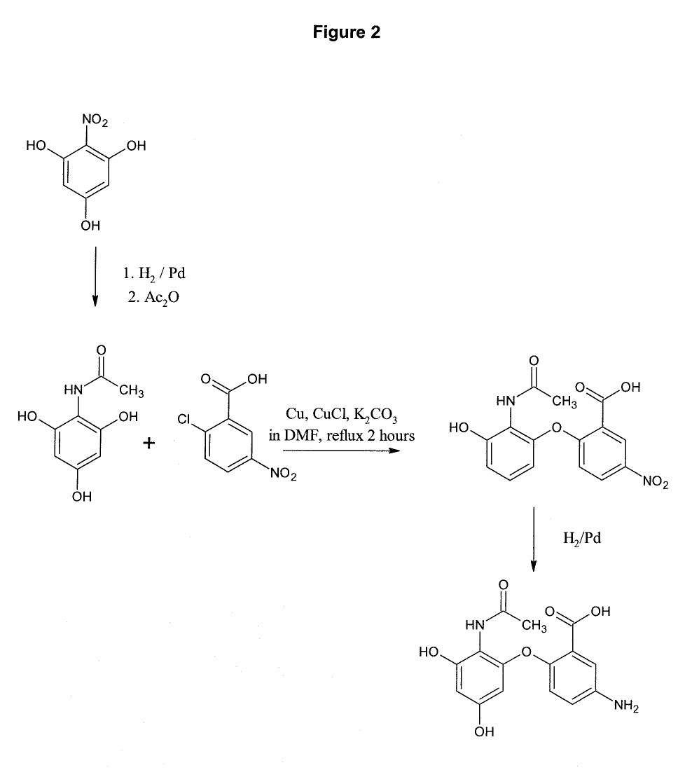

FIG. 2 Chemical synthesis of compound 88

FIG. 3 General structure (lead compound) of the activators of the hyaluronan transport/export

FIG. 4 3D model of MRP5

### EXAMPLES

The following examples illustrate the invention. These examples should not be construed as to limit the scope of this invention. The examples are included for purposes of illustration and the present invention is limited only by the claims.

### Example 1

### Assay for Hyaluronan Transport/Export Activators in Fibroblast Cell Culture

Trypsinised fibroblasts were suspended in Dulbeccos medium at 10⁵ cells/ml and 100 μl aliquots were transferred to a 96 well microtiter plate. The first row received 200 μl of the suspension and 20 μl of the activators of the invention dissolved in phosphate buffered saline at concentrations of 4 mM. A serial dilution of the activators was established by transfer of 100 μl aliquots from the first row to the following rows. All experiments were performed in duplicates. The last row did not receive any activator and served as control. The cells were incubated for 2 days at 37° C. and aliquots (5 and 20 μl) of the culture medium were used for measurement of the hyaluronan concentration in the cell culture medium by an ELISA [125]. Briefly, the wells of a 96 well Covalink-NH-microtiter plate (NUNC) were coated with 100 μl of a mixture of 100 mg/ml of hyaluronan (Healon®), 9.2 μg/ml of N-Hydroxysuccinimide-3-sulfonic acid and 615 μl/ml of 1-ethyl-3-(3-dimethylaminopropyl)-carbodiimide for 2 hours at room temperature and overnight at 4° C. The wells were washed three times with 2 M NaCl, 41 mM MgSO₄, 0.05% Tween-20 in 50 mM phosphate buffered saline pH 7.2 (buffer A) and once with 2 M NaCl, 41 mM MgSO₄, in phosphate buffered saline pH 7.2. Additional binding sites were blocked by incubation with 300 μl of 0.5% bovine serum albumin in phosphate buffered saline for 30 min at 37° C. Calibration of the assay was performed with standard concentrations of hyaluronan ranging from 15 ng/ml to 6000 ng/ml in equal volumes of culture medium as used for measurement of the cellular supernatants. A solution (50 μl) of the biotinylated hyaluronan binding fragment of aggrecan (Calbiochem) in 1.5 M NaCl, 0.3 M guanidinium hydrochloride, 0,08% bovine serum albumin 0.02% NaN₃ 25 mM phosphate buffer pH 7.0 was preincubated with 50 μl of the standard hyaluronan solutions or cellular supernatants for 1 hour at 37° C. The mixtures were transferred to the hyaluronan-coated test plate and incubated for 1 hour at 37° C. The microtiter plate was washed three times with buffer A and incubated with 100 μl/well of a solution of streptavidin-horseraddish-peroxidase (Amersham) at a dilution of 1:100 in phosphate buffered saline, 0.1% Tween-20 for 30 min at room temperature. The plate was washed five times with buffer A and the colour was developed by incubation with a 100 μl/well of a solution of 5 mg o-phenylenediamine and 5 μl 30% H₂O₂ in 10 ml of 0.1 M citrate-phosphate buffer pH 5.3 for 25 min at room temperature. The adsorption was read at 490 nm. The concentrations in the samples were calculated from a logarithmic regression curve of the hyaluronan standard solutions.

The results obtained are depicted in the table disclosed hereinbefore.

### Example 2

### Chemical Synthesis of Compound 86

A mixture of 2.5 g 2-Nitroresorcinol (16 mMol), 2.5 g 2-Chlorobenzoic acid (16 mMol) 4.5 g K₂CO₃ (32 mMol), 50 mg copper, 50 mg CuCl in 20 ml dimethylformamid was refluxed for 3 hours. After cooling to room temperature, 20 ml of concentrated HCl and 200 ml of water was added, and the product was extracted with 200 of chloroform. The organic phase was dried over Na₂SO₄ and evaporated. The product was dissolved in 20 ml of methanol, 0.1 g of palladium (10% on charcoal) was added and hydrogenated in an H₂-atmosphere overnight at room temperature. The catalyst was removed by centrifugation, and the resulting amine was N-acetylated by addition of 0.8 g of acetic anhydride for 30 min at room temperature. The reagent was evaporated and last traces were removed by evaporation with toluene to obtain compound 86.

### Example 3

### Chemical Synthesis of Compound 88

Nitrophloroglucinol (1 g, 6.5 mMol) was dissolved in 10 ml of methanol and hydrogenated in a hydrogen atmosphere in the presence of 0.1 g of 10% Pd/C overnight at room temperature. The solvent was removed by evaporation an the residue was dissoved in 12 ml of dimethylformamide. 2-chlor-5-nitrobenzoic acid (1.2 g; 6 mMol), 1.7 g of K2CO3, 0.18 g of copper powder and 0.18 g of CuCl were added and the mixture was refluxed for 3 hours. After cooling to room temperature, 12 ml of concentrated HCl and 120 ml of water were added, and the product was extracted with 120 of ethylacetate. The organic phase was dried over Na2SO4 and evaporated. The product was dissolved in 12 ml of methanol; 0.1 g of palladium (10% ob charcoal) was added and hydrogenated in a hydrogen atmosphere overnight at room temperature. The catalyst was removed by centrifugation, and the solvant was evaporated to obtain compound 1D.

### Example 4

### Chemical Synthesis of Compounds 88

Nitrophloroglucinol was catalytically reduced with H2/Pd and acetylated with acetic anhydride and pyridine. The product acetamido-phloroglycinol (16 mmol) was refluxed with 2.5 g 2-chloro-5-nitrobenzoic acid (16 mMol) 4.5 g K₂CO₃ (32 mMol), 50 mg copper, 50 mg CuCl in 20 ml dimethylformamid for 3 hours. After cooling to room temperature, 20 ml of concentrated HCl and 200 ml of water was added, and the product was extracted with 200 of chloroform. The organic phase was dried over Na₂SO₄ and evaporated. The product was dissolved in 20 ml of methanol, 0.1 g of palladium (10% on charcoal) was added and hydrogenated in an H₂-atmosphere overnight at room temperature. The catalyst was removed by centrifugation and the solvent was evaporated to obtain compound 88.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

### REFERENCES

[1] Laurent, T. C. (1964) The interaction between polysaccharides and other macromolecules. The exclusion of molecules from hyaluronic acid gels and solutions. Biochem. J, 93, 106-112.
[2] Laurent, T. C. & Gergely, L. (1955) Light scattering studies on hyaluronic acid. J Biol. Chem., 212, 325-333.
[3] Prehm, P. (1983) Synthesis of hyaluronate in differentiated teratocarcinoma cells. Mechanism of chain growth. Biochem. J., 211, 191-198.
[4] Prehm, P. (1983) Synthesis of hyaluronate in differentiated teratocarcinoma cells. Characterization of the synthase. Biochem. J., 211, 181-189.
[5] Prehm, P. (1984) Hyaluronate is synthesized at plasma membranes. Biochem. J., 220, 597-600.
[6] Prehm, P. (2006) Biosynthesis of Hyaluronan: Direction of Chain Elongation. Biochem. J., 398, 469-473.
[7] Ouskova, G., Spellerberg, B., & Prehm, P. (2004) Hyaluronan release from Streptococcus pyogenes: Export by an ABC transporter. Glycobiology, 14, 931-938.
[8] Prehm, P. & Schumacher, U. (2004) Inhibition of hyaluronan export from human fibroblasts by inhibitors of multidrug resistance transporters. Biochem. Pharmacol., 68, 1401-1410.
[9] Schulz, T., Schumacher, U., & Prehm, P. (2007) Hyaluronan export by the ABC-transporter MRP5 and its modulation by intracellular cGMP. J Biol. Chem., 282, 20999-21004.
[10] Nickel, V., Prehm, S., Lansing, M., Mausolf, A., Podbielski, A., Deutscher, J., & Prehm, P. (1998) An ectoprotein kinase of group C streptococci binds hyaluronan and regulates capsule formation. J. Biol. Chem., 273, 23668-23673.
[11] Brecht, M., Mayer, U., Schlosser, E., & Prehm, P. (1986) Increased hyaluronate synthesis is required for fibroblast detachment and mitosis. Biochem. J., 239, 445-450.
[12] Lüke, H. J. & Prehm, P. (1999) Synthesis and shedding of hyaluronan from plasma membranes of human fibroblasts and metastatic and non-metastatic melanoma cells. Biochem. J., 343, 71-75.
[13] Prehm, P. (2005) Inhibitors of hyaluronan export prevent proteoglycan loss from osteoarthritic cartilage. J. Rheumatol., 32, 690-696.
[14] Deiters, B. & Prehm, P. (2008) Inhibition of hyaluronan export reduces collagen degradation in IL-1 treated cartilage. Arthritis Res. Ther., 10, R8.
[15] Underhill, C. B. & Toole, B. P. (1979) Binding of hyaluronate to the surface of cultured cells. J. Cell Biol., 82, 475-484.
[16] Underhill, C. B. & Toole, B. P. (1982) Transformation-dependent loss of the hyaluronate-containing coats of cultured cells. J. Cell Physiol., 110, 123-128
[29] Dube, B., Luke, H. J., Aumailley, M., & Prehm, P. (2001) Hyaluronan reduces migration and proliferation in CHO cells. Biochim. Biophys. Acta, 1538, 283-289.
[30] Fraser, J. R. & Laurent, T. C. (1989) Turnover and metabolism of hyaluronan. Ciba. Found. Symp., 143, 41-53.
[31] Lebel, L., Gabrielsson, J., Laurent, T. C., & Gerdin, B. (1994) Kinetics of circulating hyaluronan in humans. Eur. J. Clin. Invest., 24, 621-626.
[32] Reed, R. K., Laurent, T. C., & Taylor, A. E. (1990) Hyaluronan in prenodal lymph from skin: changes with lymph flow. Am. J. Physiol., 259, H1097-100.
[33] Coleman, P. J., Scott, D., Mason, R. M., & Levick, J. R. (2000) Role of hyaluronan chain length in buffering interstitial flow across synovium in rabbits. J. Physiol., 526, 425-434.
[34] Laurent, T. C., Dahl, L. B., & Lilja, K. (1993) Hyaluronan injected in the anterior chamber of the eye is catabolized in the liver. Exp. Eye Res., 57, 435-440.
[125] Stern, M. & Stern, R. (1992) An ELISA-like assay for hyaluronidase and hyaluronidaseinhibitors. Matrix, 12, 397-403.
[260] Gresele, P. & Momi, S. (2006) Pharmacologic profile and therapeutic potential of NCX 4016, a nitric oxide-releasing aspirin, for cardiovascular disorders. Cardiovasc. Drug Rev., 24, 148-168.
[25a] Prehm, P. (1985) Inhibition of hyaluronate synthesis. Biochem. J., 225, 699-705.
[92a] Stein, W. D. (1997) Kinetics of the multidrug transporter (P-glycoprotein) and its reversal. Physiol Rev., 77, 545-590.
[93a] Twentyman, P. R., Rhodes, T., & Rayner, S. (1994) A comparison of rhodamine 123 accumulation and efflux in cells with P-glycoprotein-mediated and MRP-associated multidrug resistance phenotypes. Eur. J. Cancer, 30A, 1360-1369.
[87a] Stern, M. & Stern, R. (1992) An ELISA-like assay for hyaluronidase and hyaluronidaseinhibitors. Matrix, 12, 397-403.
[89a] Lüke, H. J. & Prehm, P. (1999) Synthesis and shedding of hyaluronan from plasma membranes of human fibroblasts and metastatic and non-metastatic melanoma cells. Biochem. J., 343, 71-75.
[91a] Alberts, B., Bray, D., Lewis, J., Raff, M., Roberts, K., & Watson, J. D. (2002) Molecular Biology of the Cell, 3 edn. Garland, New York.
[92a] Stein, W. D. (1997) Kinetics of the multidrug transporter (P-glycoprotein) and its reversal. Physiol Rev., 77, 545-590.
[93a] Twentyman, P. R., Rhodes, T., & Rayner, S. (1994) A comparison of rhodamine 123 accumulation and efflux in cells with P-glycoprotein-mediated and MRP-associated multidrug resistance phenotypes. Eur. J. Cancer, 30A, 1360-1369.
[96a] Cooper, C. (1998) Osteoarthritis and related disorders. Epidemiology. In Rheumatology (Klippel, J. H. & Dieppe, P. A., eds), pp. 2.1-2.8. Mosby, London.
[99a] Stern, M. & Stern, R. (1992) An ELISA-like assay for hyaluronidase and hyaluronidaseinhibitors. Matrix, 12, 397-403.
[100a] de Belder, A. N. & Wik, K. O. (1975) Preparation and properties of fluorescein-labelled hyaluronate. Carbohydr. Res., 44, 251-257.
[101a] Rao, C. M., Jilani, A., Swarnakar, S., Deb, T. B., & Datta, K. (1996) A method to radioiodinate hyaluronic acid and its use as a probe to detect hyaluronic acid-binding proteins. J. Biol. Chem., 255, 7218-7224.
[102a] Termeer, C. C., Hennies, J., Voith, U., Ahrens, T., Weiss, M., Prehm, P., & Simon, J. C. (2000) Oligosaccharides of hyaluronan are potent activators of dendritic cells. J. Immunol., 165, 1863-1870.
[103a] Prehm, P. & Scheid, A. (1978) Sensitive method for the analysis of carbohydrates by gas chromatography of 3H-labeled alditol acetates. J. Chromatogr., 166, 461-467.
[117a] Asplund, T., Versnel, M. A., Laurent, T. C., & Heldin, P. (1993) Human mesothelioma cells produce factors that stimulate the production of hyaluronan by mesothelial cells and fibroblasts. Cancer Res., 53, 388-392.
[118a] Jacobson, A., Rahmanian, M., Rubin, K., & Heldin, P. (2002) Expression of hyaluronan synthase 2 or hyaluronidase 1 differentially affect the growth rate of transplantable colon carcinoma cell tumors. Int. J. Cancer, 102, 212-219.
[119a] Tufveson, G., Hallgren, R., Johnsson, C., & Wahlberg, J. Use of hyaluronidase in treatment of interstitial edema from organ grafts. Tufveson, Gunnar, Hallgren, Roger, Johnsson, Cecilia, and Wahlberg, January [WO 9808538],
[120a] Engstrom Laurent, A., Feltelius, N., Hallgren, R., & Wasteson, A. (1985) Raised serum hyaluronate levels in scleroderma: an effect of growth factor induced activation of connective tissue cells? Ann. Rheum. Dis., 44, 614-620.
[121a] Lundin, A., Engstrom Laurent, A., Hallgren, R., & Michaelsson, G. (1985) Circulating hyaluronate in psoriasis. Br. J. Dermatol., 112, 663-671.
[122a] Hallgren, R., Eklund, A., Engstrom Laurent, A., & Schmekel, B. (1985) Hyaluronate in bronchoalveolar lavage fluid: a new markerin sarcoidosis reflecting pulmonary disease. Br. Med. J. Clin. Res. Ed., 290, 1778-1781.
[123a] Eklund, A., Hallgren, R., Blaschke, E., Engstrom Laurent, A. P.-U., & Svane, B. (1987) Hyaluronate in bronchoalveolar lavage fluid in sarcoidosis and its relationship to alveolar cell populations. Eur. J. Respir. Dis., 71, 30-36.
[124a] Nettelbladt, O. & Hallgren, R. (1989) Hyaluronan (hyaluronic acid) in bronchoalveolar lavage fluid during the development of bleomycin-induced alveolitis in therat. Am. Rev. Respir. Dis., 140, 1028-1032.
[125a] Nettelbladt, O., Lundberg, K., Tengblad, A., & Hallgren, R. (1990) Accumulation of hyaluronan in bronchoalveolar lavage fluid is independent of iron-, complement- and granulocyte-depletionin bleomycin-induced alveolitis in the rat. Eur. Respir. J., 3, 765-771.
[126a] Hallgren, R., Gerdin, B., Tengblad, A., & Tufveson, G. (1990) Accumulation of hyaluronan (hyaluronic acid) in myocardial interstitial tissue parallels development of transplantation edema in heart allografts in rats. J. Clin. Invest, 85, 668-673.
[127a] Nettelbladt, O., Scheynius, A., Bergh, J., Tengblad, A., & Hallgren, R. (1991) Alveolar accumulation of hyaluronan and alveolar cellular response in bleomycin-induced alveolitis. Eur. Respir. J., 4, 407-414.
[128a] Bjermer, L., Hallgren, R., Nilsson, K., Franzen, L., Sandstrom, T. S.-B., & Henriksson, R. (1992) Radiation-induced increase in hyaluronan and fibronectin inbronchoalveolar lavage fluid from breast cancer patients is suppressed by smoking. Eur. Respir. J., 5, 785-790.
[129a] Ahrenstedt, O., Knutson, L., Hallgren, R., & Gerdin, B. (1992) Increased luminal release of hyaluronan in uninvolved jejunum in active Crohn's disease but not in inactive disease or in relatives. Digestion, 52, 6-12.
[130a] Johnsson, C., Hallgren, R., & Tufveson, G. (1993) Recovery of hyaluronan during perfusion of small bowel transplantation reflects rejection. Transplantation, 55, 477-479.
[131a] Waldenstrom, A., Fohlman, J., Ilback, N. G., Ronquist, G., & Hallgren, R. G.-B. (1993) Coxsackie B3 myocarditis induces a decrease in energy charge and accumulation of hyaluronan in the mouse heart. Eur. J. Clin. Invest., 23, 277-282.
[132a] Wells, A., Larsson, E., Hanas, E., Laurent, T., & Hallgren, R. T.-G. (1993) Increased hyaluronan in acutely rejecting human kidney grafts. Transplantation, 55, 1346-1349.
[133a] Tufveson, G., Hallgren, R., Johnsson, C., & Wahlberg, J. Use of hyaluronidase in treatment of interstitial edema from organ grafts. Tufveson, Gunnar, Hallgren, Roger, Johnsson, Cecilia, and Wahlberg, January WO 97-SE1313 [WO 9808538 A1]. 1998. PCT Int. Appl., 18 pp. CODEN: PIXXD2. Jul. 24, 1997. Ref Type: Patent
[134a] Johnsson, C., Hallgren, R., Elvin, A., Gerdin, B., & Tufveson, G. (1999) Hyaluronidase ameliorates rejection-induced edema. TRANSPLANT INTERNATIONAL, 12, 235-243.
[135a] Johnsson, C., Hallgren, R., & Tufveson, G. (2000) Role of hyaluronan in acute pancreatitis. Surgery, 127, 650-658.
[136a] Kimata, K., Honma, Y., Okayama, M., Oguri, K., Hozumi, M., & Suzuki, S. (1983) Increased synthesis of hyaluronic acid by mouse mammary carcinoma cell variants with high metastatic potential. Cancer Res., 43, 1347-1354.
[137a] Zhang, L., Underhill, C. B., & Chen, L. (1995) Hyaluronan on the surface of tumor cells is correlated with metastatic behavior. Cancer Res., 55, 428-433.
[138a] West, D. C. & Shaw, D. M. (1998) Tumour hyaluronan in relation to angiogenesis and metastasis. In The chemistry, biology and medical applications of hyaluronan and its derivatives (Laurent, T. C., ed), pp. 227-233. Portland Press, London.
[139a] Simpson, M. A., Reiland, J., Burger, S. R., Furcht, L. T., Spicer, A. P., Oegema, T. R., Jr., & McCarthy, J. B. (2001) Hyaluronan Synthase Elevation in Metastatic Prostate Carcinoma Cells Correlates with Hyaluronan Surface Retention, a Prerequisite for Rapid Adhesion to Bone Marrow Endothelial Cells. J. Biol. Chem., 276, 17949-17957.
[140a] Knudson, W. (1996) Tumor-associated hyaluronan—Providing an extracellular matrix that facilitates invasion. Am. J. Pathol., 148, 1721-1726.
[143a] Schinkel, A. H. & Jonker, J. W. (2003) Mammalian drug efflux transporters of the ATP binding cassette (ABC) family: an overview. Adv. Drug Deliv. Rev., 55, 3-29.

1. A compound (activator) which is characterized by a formula selected from the following formulas A, B and/or C 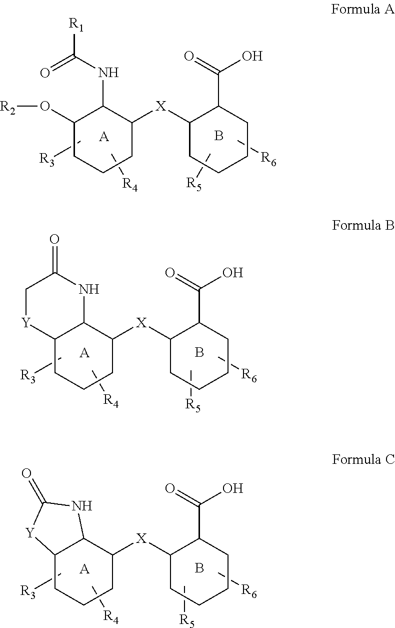
or a pharmaceutically acceptable salt thereof,
wherein
the ring systems A and B are independently selected from a monosaccharide, aryl (preferably phenyl), a heteroaryl or cycloalkyl (preferably cyclohexan), preferably with all substituents in equatorial configurations;
R1 is independently selected from alkyl (preferably C1 to C6), a substituted or unsubstituted phenyl, preferably CH3;
R2 is H, alkyl (preferably C1 to C6), a carbohydrate in a glycosidic β-linkage, preferably H;
R3, R4, R5, and R6 are independently selected from H, (OH) hydroxy, alkyl preferably C1 to C6, alkoxy (preferably C1 to C6), amino, alkylamino (preferably C1 to C6), halogen, benzylamino, or benzoylamino;
X is O, NH, alkylamino (NR), CO, S; and
Y is O, NH, alkylamino (NR), CO, S.

2. A pharmaceutical composition comprising one or more compound(s) selected from the following formula A, B and/or C of claim 1 and, optionally, a pharmaceutically acceptable carrier.

3. A method for the treatment of (for treating) and/or preventing diseases or medical conditions which benefit from an increased transport of hyaluronan across a lipid bilayer comprising a step of administering one or more compound(s) selected from the following formula A, B and/or C of claim 1 to a subject.

4. The pharmaceutical composition of claim 2 for the treatment of (for treating) and/or preventing diseases or medical conditions which benefit from an increased transport of hyaluronan across a lipid bilayer.

5. The method of claim 3 wherein said disease is associated with or characterized by an decreased transport/export of hyaluronan of cells, selected from the group consisting of fibroblasts, sarcomas, carcinomas, smooth muscle cells, endothelial cells, endodermal cells, liver stellate cells, mesothelioma cells, melanoma cells, oligodendroglial cells, glioma cells, Schwann cells, synovial cells, myocaridal cells, trabecular-meshwork cells, cumulus cells, liver adipocytes (Ito cells), keratinocytes, epithelial cells and/or chondrocytes.

6. The method of claim 5, wherein said cell is comprised in a tissue.

7. The method of claim 6, wherein said cell or said tissue is derived from a mammalian subject.

8. The method of claim 7, wherein said mammalian subject is a human, a horse, a camel, a dog, a cat, a pig, a cow, or a goat.

9. The method of claim 3 for the treatment of psoriasis, acne, aged wrinkled skin, wound healing, cystic fibrosis and/or scareless healing.

10. The method of claim 3, wherein said activator(s) is(are) to be administered prophylactically.

11. The method of claim 3, wherein said activator(s) is(are) administered therapeutically.

12. A method for manufacturing a pharmaceutical composition comprising a step of formulating one or more compound(s) selected from the following formula A, B and/or C of claim 1 in a pharmaceutically acceptable form.

13. A cosmetic composition comprising one or more compound(s) selected from the following formula A, B and/or C of claim 1.

14. A method for increasing the attractiveness of skin comprising a step of applying one or more compound(s) selected from the following formula A, B and/or C of claim 1 to skin.
